(19) United States
(12) Patent Application Publication
(10) Pub. No.: US 2021/0338300 A1
(43) Pub. Date: Nov. 4, 2021

(54) CABLE CRIMP CAP APPARATUS AND METHOD

(71) Applicant: Little Engine, LLC, Belmont, NC (US)

(72) Inventors: J. Dean Cole, Orlando, FL (US); Franz W. Kellar, Gastonia, NC (US); Michael D. Bissette, Belmont, NC (US)

(21) Appl. No.: 17/264,198

(22) PCT Filed: Jul. 31, 2019
(86) PCT No.: PCT/US2019/044477

§ 371 Date: Jan. 28, 2021

Related U.S. Application Data
(60) Provisional application No. 62/715,532, filed on Aug. 7, 2018.
(60) Provisional application No. 62/712,300, filed on Jul. 31, 2018.

(51) Int. Cl. A61B 17/88; A61B 17/04; A61B 17/84

(52) U.S. Cl. CPC A61B 17/8869; A61B 2017/00477; A61B 17/842; A61B 17/0487

(57) ABSTRACT

A crimp cap apparatus includes: a crimp sleeve having a peripheral wall defining an exterior surface and an interior passage sized to receive a tensile member having a predetermined outer diameter, the crimp sleeve being configured such that it can be crimped around the tensile member by an application of mechanical force which reduces its dimensions from a first size to a second, smaller size; and a cap having a bottom surface, a convex top surface, a central bore extending from the bottom surface to the top surface for receiving the crimp sleeve, wherein the cap includes a clamping structure disposed in the central bore and configured to permit elastic deflection in response to being pressed over the crimp sleeve the crimp sleeve and apply a clamping force there-to to secure the cap in place.

### BACKGROUND OF THE INVENTION

This invention relates generally to medical implants, and more particularly to apparatus used to apply tension along or across a ligament to repair, augment, or replace it, or applying tension across a bone fracture to reduce it.

Medical implants for tensioning purposes typically comprise one or more tensile members (e.g., sutures or orthopedic cables) connected to one or more anchors (e.g., suture anchors or suture locks) to create a converging tensile force between the two anchors. This general concept has been used in the orthopedic and sports medicine fields to repair torn or damaged tendons and ligaments, to replace missing or displaced tendons and ligaments, and to anchor grafted or artificial tendons and ligaments to bones so that they can grow back together.

One particular type of tensile member anchor includes a crimp sleeve which is crimped around a distal end of a tensile member to secure it in place. One problem with prior art sleeves is that they can leave a leave a sharp and/or protruding end after crimping and cable cutting, resulting in a crimped end that can create soft tissue irritation. The sharpness and irritating features of the prior art devices include both the crimp edge and the frayed end of the cable itself.

### BRIEF SUMMARY OF THE INVENTION

This problem is addressed by a crimp cap which is effective to cover cable ends and protect soft tissue.

According to one aspect of the technology described herein, a crimp cap apparatus includes: a crimp sleeve having a peripheral wall defining an exterior surface and an interior passage sized to receive a tensile member having a predetermined outer diameter, the crimp sleeve being configured such that it can be crimped around the tensile member by an application of mechanical force which reduces its dimensions from a first size to a second, smaller size; and a cap having a bottom surface, a convex top surface, a central bore extending from the bottom surface to the top surface for receiving the crimp sleeve, wherein the cap includes a clamping structure disposed in the central bore and configured to permit elastic deflection in response to being pressed over the crimp sleeve the crimp sleeve and apply a clamping force thereto to secure the cap in place.

According to another aspect of the technology described herein, a method of securing a tensile member includes: fitting a washer having a central bore over a tensile member; fitting a crimp sleeve over the tensile member abutting the washer, wherein the crimp sleeve has a peripheral wall defining an outer surface and an interior passage sized to receive the tensile member; crimping the crimp sleeve around the tensile member by an application of mechanical force so as to reduce its dimensions from a first size to a second, smaller size; providing a cap having a bottom surface, a convex top surface, a central bore extending from the bottom surface to the top surface for receiving the crimp sleeve, wherein the cap includes a clamping structure disposed in the central bore; and pressing the cap over the crimp sleeve such that the clamping structure deflects elastically and applies a clamping force to the crimp sleeve to secure the cap in place.

### BRIEF DESCRIPTION OF THE DRAWINGS

The invention may be best understood by reference to the following description taken in conjunction with the accompanying drawing figures in which:

FIG. 1 is a side view of a section of a tensile member;

FIG. 2 is an end view of the tensile member of FIG. 1;

FIG. 3 is a side elevation view of a crimp sleeve;

FIG. 4 is a perspective view of the crimp sleeve of FIG. 3;

FIG. 5 is an end view of the crimp sleeve of FIG. 3;

FIG. 6 is a perspective view of a washer;

FIG. 7 is a perspective view of an assembly of a tensile member, washer, and crimp sleeve;

FIG. 8 is a perspective view of a first embodiment of a cap;

FIG. 9 is a cross-sectional view of the cap of FIG. 8;

FIG. 10 is a cross-sectional view of an assembly of the cap of FIG. 8 to a tensile member and crimp sleeve;

FIG. 11 is a schematic side view showing a tensile member routed through a washer and crimp sleeve;

FIG. 12 is a schematic side view showing a crimp sleeve and washer crimped to a tensile member;

FIG. 13 is a schematic side view showing installation of a cap over a tensile member, crimp sleeve, and washer;

FIG. 14 is a schematic side view showing a cap pressed into final position over a tensile member, crimp sleeve, and washer;

FIG. 15 is a cross-sectional view of the assembly of FIG. 14;

FIG. 16 is a schematic side view showing a tool being used to compress a cap;

FIG. 17 is a schematic side view showing an assembly of a tensile member, washer, crimp sleeve, and cap, with the tensile member being trimmed below the surface of the cap;

FIG. 18 is a perspective view of the crimp sleeve having notches formed therein;

FIG. 19 is a cross-sectional view of a cap installed over the crimp sleeve of FIG. 19;

FIG. 20 is a perspective view of an alternative embodiment of a cap;

FIG. 21 is a top plan view of the cap of FIG. 20;

FIG. 22 is a cross-sectional view of the cap of FIG. 20;

FIG. 23 is a cross-sectional view of the cap of FIG. 20 assembled to a tensile member, washer, and crimp sleeve;

FIG. 24 is a perspective view of an alternative embodiment of a cap;

FIG. 25 is a cross-sectional view of the cap of FIG. 24;

FIG. 26 is a partially-sectioned top view of an alternative embodiment of a cap;

FIG. 27 is a perspective view of the cap of FIG. 26;

FIG. 28 is a cross-sectional view of the cap of FIG. 26;

FIG. 29 is a perspective, partially-sectioned view of an alternative embodiment of a cap;

FIG. 30 is a cross-sectional view of the cap of FIG. 29;

FIG. 31 is a perspective exploded view of a tensile member, washer, crimp sleeve, and cap with a suture threaded through holes therein; and

FIG. 32 is a perspective view of the assembly of FIG. thirty, with the suture pulled down to cinch the components together.

### DETAILED DESCRIPTION OF THE INVENTION

In general, the technology described herein provides a cap and crimp system and method that enables provisional and permanently stable tensioning of a tensile member while shielding the cut end of the tensile member.

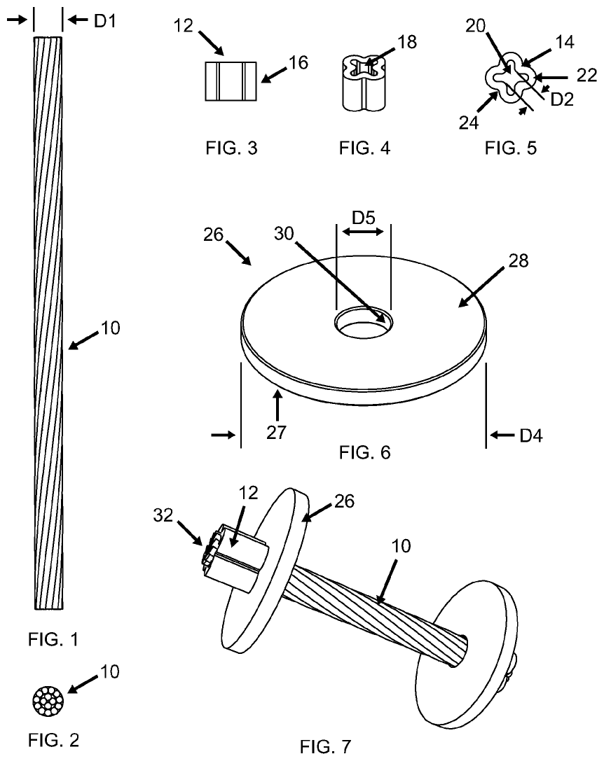

The anchor, installation system, and installation method described herein are suitable for receiving and securing a tensile member to bone. The term “tensile member” as used herein generally refers to any flexible element capable of transmitting a tensile force, such as a filament, thread, or cable. Nonlimiting examples of known types of tensile members include sutures and orthopedic cables. FIGS. 1 and 2 illustrate a short segment of a representative tensile member 10 having an outside diameter “D1”. Commercially-available tensile members intended to be implanted in the human body may have a diameter “D1” ranging from tens of microns in diameter to multiple millimeters in diameter. Commercially-available tensile members may be made from a variety of materials such as polymers or metal alloys. Nonlimiting examples of suitable materials include absorbable polymers, nylon, ultrahigh molecular weight polyethylene (“UHMWPE”) or polypropylene titanium alloys, or stainless steel alloys. Known physical configurations of tensile members include monofilament, braided, twisted, woven, and wrapped.

FIGS. 3-5 illustrate an exemplary embodiment of a crimp sleeve 12. The crimp sleeve 12 is a short tube-like element with the peripheral wall 14. The peripheral wall 14 defines an exterior surface 16 and an interior surface 18 circumscribing an interior passage 20. The peripheral wall 14 may have numerous cross-sectional shapes such as circular, elliptical, polygonal, or lobed. In the illustrated example, the peripheral wall 14 has a lobed shape with a circumferential array of outwardly-protruding lobes 22 alternating with depressions 24. As manufactured and prior to use, the interior passage 20 is a minimum internal dimension “D2” which is selected to be able to pass over the outer diameter D1 of the tensile member 10.

The crimp sleeve 12 is configured in terms of its material, shape, and dimensions such that it can be swaged or crimped around the tensile member 10. By the application of mechanical force, reducing its exterior and interior dimensions from a first size to a second smaller size. For example, it may be made from a material which is plastically deformable. Nonlimiting examples of suitable materials include polymers and metal alloys. The crimp sleeve 12 may be made from a biocompatible material, defined as material which causes little or no damage to living tissue.

FIG. 6 illustrates an exemplary washer 26. The washer 26 is of conventional construction with an outer diameter “D4”, opposed first and second faces 27, 28, a selected thickness, and a central bore 30 having a diameter “D5” which is selected to be able to pass over the outer diameter D1 of the tensile member 10.

FIG. 7 illustrates an assembly of the tensile member 10, crimp sleeve 12, and washer 26. The tensile member 10 passes through the central bore 30 of the washer 26 and the interior passage 20 of the crimp sleeve 12. The crimp sleeve 12 abuts the washer 26. The crimp sleeve 12 is swaged or crimped by the application of external force, reducing the initial minimum internal dimension “D2” such that the crimp sleeve 12 tightly engages the tensile member 10 so that tension can be maintained therein. In this illustration, a distal end 32 of the tensile member 10 has been cut or trimmed; however, a small portion of it extends beyond the end of the crimp sleeve 12.

According to the principles of the present invention, a cap may be provided which can be connected to the crimp sleeve 12, covering it to provide a smooth surface which will prevent injury or irritation to the patient or surgeon.

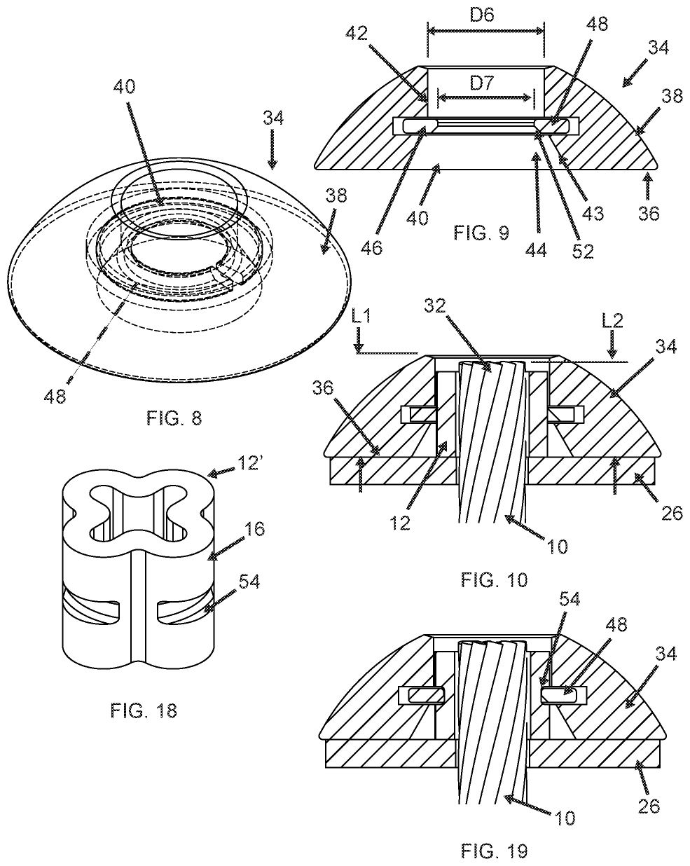

FIGS. 8 and 9 illustrate an exemplary cap 34. The cap 34 has a generally flat bottom surface 36 and an opposed top surface 38 which is convex or domed. The overall shape of the cap 34 may be circular in plan view as shown, or oblong, or some other convenient shape. The cap 34 includes a central bore 40 passing therethrough from the bottom surface 36 to the top surface 38. The central bore 40 has a first portion 42 with a diameter “D6” sufficiently large to pass over the exterior surface 16 of the crimp sleeve 12 in the crimped condition. Optionally, the central bore 40 may include a second portion 43 adjacent the bottom surface 36 which is enlarged or tapered.

The cap 34 may be made from any suitable biocompatible material, for example a polymer.

The cap 34 includes a clamping structure 44 disposed in the central bore 40. The clamping structure 44 has a central opening 46 with an initial size “D7” which is smaller than an exterior surface 16 of the crimp sleeve 12 in the crimped condition. The clamping structure 44 is configured to permit elastic deflection so that it can pass over the crimp sleeve 12 and apply a clamping force thereto to secure the cap 34 in place.

In the embodiment shown in FIGS. 8 and 9, the clamping structure 44 comprises a split snap ring 48 which is received in a circumferential groove 50 formed in the central bore 40. The split snap ring 48 is elastically deflectable and may include a chamfer or lead-in surface 52. It may be made, for example, from a material such as a metal alloy.

FIG. 10 shows a tensile member 10 having a washer 26, crimp sleeve 12, and the cap 34 of FIG. 5 assembled thereto. It can be seen that the bottom surface 36 abuts the washer 26. The distance from the bottom surface 36 to the top surface 38 at the central bore 40 (designated “L1”) is greater than a distance from the washer 26 to the distal end 32 of the tensile member 10 (designated “L2”).

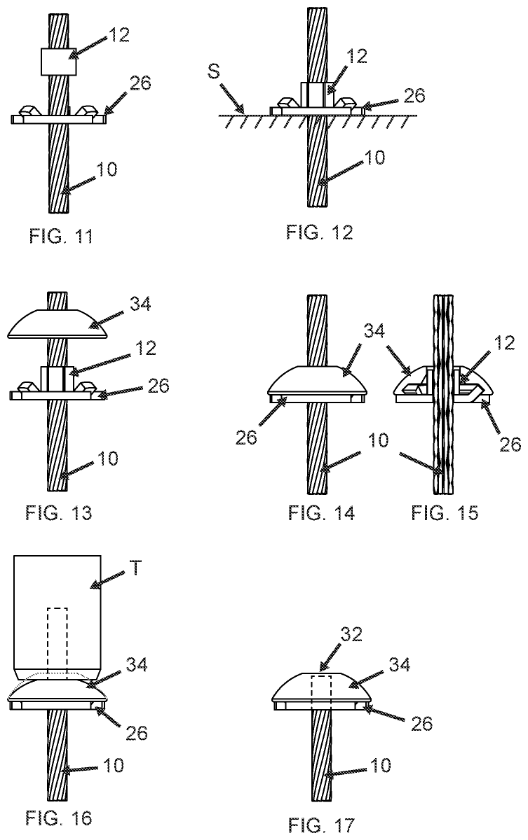

FIGS. 11-17 show the steps in completing the assembly of FIG. 10. Initially, the tensile member 10 would be routed through passage formed in a bone or other body structure (not shown). The washer 26 and crimp sleeve 12 are slid over the tensile member 10 (see FIG. 11).

Referring to FIG. 12, the tensile member 10 is tensioned along its axis while the washer 26 and the crimp sleeve 12 are pushed firmly against a bone surface (shown schematically at “S”). The crimp sleeve 12 is then crimped or swaged while the tensile member 10 is under the desired tension, leaving a substantial length of the tensile member 10 extending past a crimp sleeve 12.

Referring to FIG. 13, the cap 34 is slid over the tensile member 10 and pressed down firmly so that the clamping structure 44 engages the crimp sleeve 12, retaining the cap 34 in place as seen in FIGS. 14 and 15.

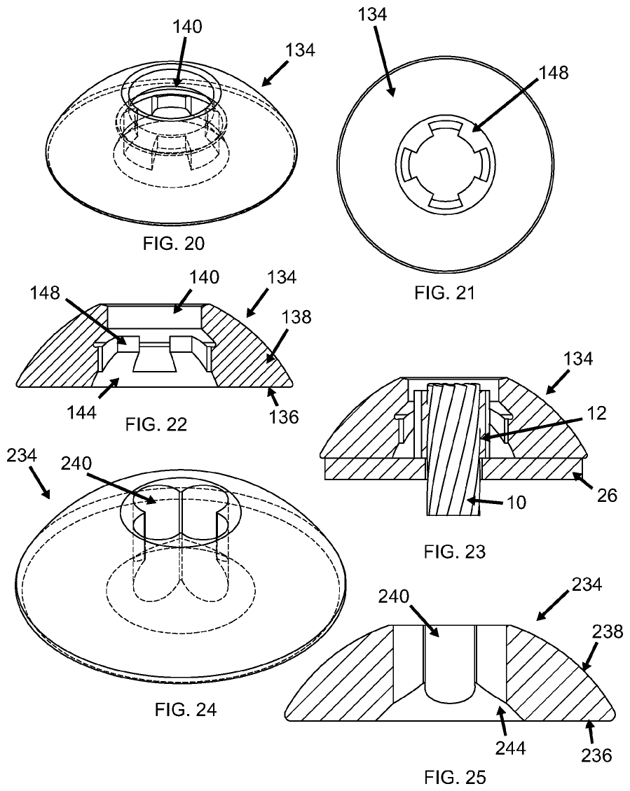

Referring to FIG. 16, the cap 34 is then pressed down using an appropriate tool “T” to compress it, and the tensile member 10 is cut off as close as possible to the end of the crimp sleeve 12. Pressure is then released, allowing the cap 34 to bring back to its initial position, shielding the distal end 32 of the tensile member 10, as seen in FIG. 17.

Numerous variations are possible for the clamping structure of the cap and the mating structure of the crimp sleeve 12. For example, FIG. 18 illustrates a variation of a crimp sleeve 12′ which is mostly identical to crimp sleeve 12 described above, but which additionally has at least one notch 54 formed in the lobes 22. FIG. 19 shows this crimp sleeve 12′ assembled to a tensile member 10, washer 26, and cap 34. It can be seen that the notches 54 permit the split snap ring 48 to spring inward and engage the notches 54, providing a more secure engagement between the cap 34 and the crimp sleeve 12′.

FIGS. 20-22 illustrate an alternative cap 134 similar in overall construction to the cap 34. Elements of the cap 134 not explicitly described may be taken to be identical to corresponding structures of the cap 34. The cap 134 has a generally flat bottom surface 136, opposed top surface 138, and central bore 140.

The cap 134 includes a clamping structure 144 disposed in the central bore 140. The clamping structure 144 is configured to permit elastic deflection so that it can pass over the crimp sleeve 12 and apply a clamping force thereto to secure the cap 134 in place. In this embodiment the clamping structure 144 comprises a circumferential array of resilient barbs 148 which extend inward from the central bore 140. The resilient barbs 148 are chamfered or tapered so as to permit easy insertion of the tensile member 10 but to prevent withdrawal. FIG. 23 shows a tensile member 10 having a washer 26, crimp sleeve 12, and the cap 134 of FIG. 20 assembled thereto.

FIGS. 24 and 25 illustrate an alternative cap 234 similar in overall construction to the cap 34. Elements of the cap 234 not explicitly described may be taken to be identical to corresponding structures of the cap 34. The cap 234 has a generally flat bottom surface 236, opposed top surface 238, and central bore 240.

The cap 234 includes a clamping structure 244 disposed in the central bore 240. The clamping structure 244 is configured to permit elastic deflection so that it can pass over the crimp sleeve 12 and apply a clamping force thereto to secure the cap 234 in place. In this embodiment the clamping structure 244 comprises a shaping of the central bore 240 in a shape which is complementary to the shape of the exterior surface 16 of the crimp sleeve 12. The initial dimensions of the clamping structure 244 may be slightly smaller than the exterior surface 16 of the crimp sleeve 12 in the current condition to provide a slight interference fit.

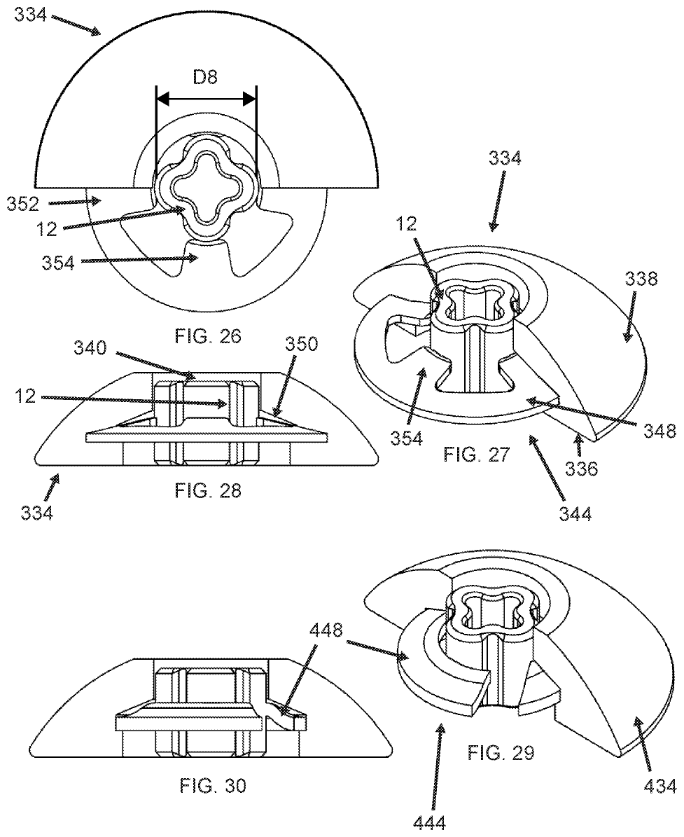

FIGS. 26-28 illustrate an alternative cap 334 similar in overall construction to the cap 34. Elements of the cap 334 not explicitly described may be taken to be identical to corresponding structures of the cap 34. The cap 334 has a generally flat bottom surface 336, opposed top surface 338, and central bore 340.

The cap 334 includes a clamping structure 344 disposed in the central bore 340. The clamping structure 344 is configured to permit elastic deflection so that it can pass over the crimp sleeve 12 and apply a clamping force thereto to secure the cap 334 in place.

In the embodiment shown in FIG. 26, the clamping structure 344 comprises a continuous snap ring 348 which is received in a circumferential groove 350 formed in the central bore 340 which is tapered or conical to permit some axial movement of the radially inner portion of the snap ring 348. The snap ring 348 has a continuous outer portion 352 and a circumferential array of inner tabs 354 which extend inward from the outer portion 352. They define a diameter “D8” smaller than the exterior surface 16 of the crimp sleeve 12 in the crimp condition. In use, the inner tabs 354 will deflect in bending to accommodate the crimp sleeve 12, but “bite into” the surface 16 in the opposite direction, preventing loss of tension on the tensile member 10.

FIGS. 29 and 30 show an alternate clamping structure 444 which may be used with a cap 434 similar to cap 334. The clamping structure 444 comprises a split snap ring 448 which has a conical shape. In use, it can deflect to provide a one-way clamping action similar to snap ring 348.

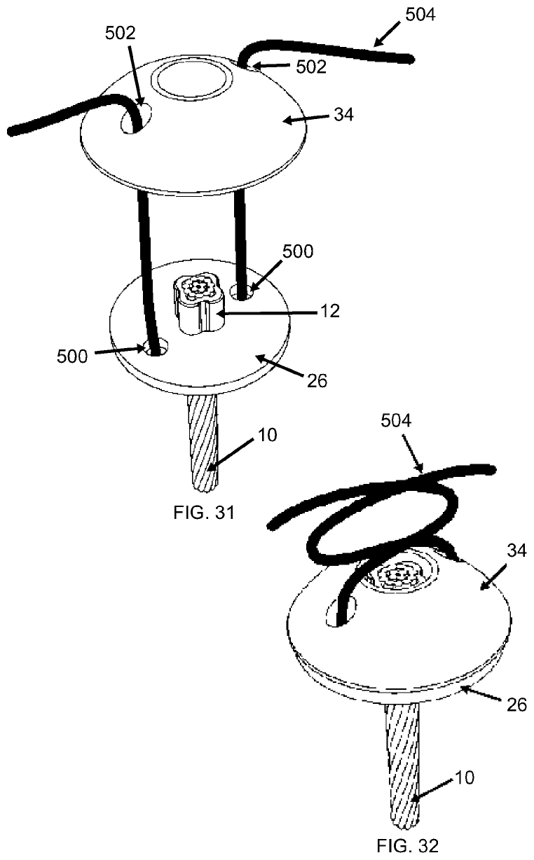

FIGS. 31 and 32 show an optional feature which may be incorporated into the cap 34 (or any of the other embodiments) and washer 26. The washer 26 includes suture holes 500 that are aligned with (e.g. are concentric to) corresponding suture holes 502 formed in the cap 34. As seen in FIG. 31, a suture 504 can be threaded through the holes 500, 502. The suture 504 can then be knotted or drawn down to secure the components together and/or to form an anchor point for additional sutures or other surgical connections.

The properties of the apparatus described herein enable provisional and permanently stable tensioning of the tensile member 10, and allows the surgeon to load-cycle and re-tension the tensile member 10 before setting final tension.

Furthermore, the cap shields the end of the tensile member 10 so that it does not protrude beyond the anchor once the swaging operation is completed. This avoids injury or irritation to the patient.

The foregoing has described a crimp cap assembly and a method for its use. While specific embodiments of the present invention have been described, it will be apparent to those skilled in the art that various modifications thereto can be made without departing from the spirit and scope of the invention. Accordingly, the foregoing description of the preferred embodiment of the invention and the best mode for practicing the invention are provided for the purpose of illustration only and not for the purpose of limitation.

1. A crimp cap apparatus, comprising:
a crimp sleeve having a peripheral wall defining an exterior surface and an interior passage sized to receive a tensile member having a predetermined outer diameter, the crimp sleeve being configured such that it can be crimped around the tensile member by an application of mechanical force which reduces its dimensions from a first size to a second, smaller size; and
a cap having a bottom surface, a convex top surface, a central bore extending from the bottom surface to the top surface for receiving the crimp sleeve, wherein the cap includes a clamping structure disposed in the central bore and configured to permit elastic deflection in response to being pressed over the crimp sleeve and to apply a clamping force to the crimp sleeve to secure the cap in place.

2. The apparatus of claim 1 wherein the clamping structure comprises a split snap ring received in a groove formed in the central bore of the cap.

3. The apparatus of claim 2 wherein the split snap ring includes a chamfered surface.

4. The apparatus of claim 2 wherein the crimp sleeve includes at least one notch formed in the exterior surface thereof, sized to receive the split snap ring.

5. The apparatus of claim 1 wherein the peripheral wall includes a circumferential array of lobes.

6. The apparatus of claim 1 wherein the clamping structure comprises a circumferential array of resilient barbs which extend inward from the central bore.

7. The apparatus of claim 1 wherein the clamping structure comprises the central bore having a shape complementary to the exterior surface of the crimp sleeve.

8. The apparatus of claim 1 where the clamping structure comprises a snap ring received in a groove formed in the central bore, wherein the groove has a conical shape.

9. The apparatus of claim 8 wherein the snap ring has a continuous outer portion and a circumferential array of inner tabs which extend inward from the outer portion.

10. The apparatus of claim 8 wherein the snap ring is a split snap ring having a conical shape.

11. The apparatus of claim 1 in combination with:
a tensile member; and
a washer having a central bore which is fitted over the tensile member and which abuts the bottom surface of the cap.

12. The apparatus of claim 11 wherein the washer and the cap include coaxially aligned suture holes.

13. A method of securing a tensile member, comprising:
fitting a washer having a central bore over a tensile member;
fitting a crimp sleeve over the tensile member abutting the washer, wherein the
crimp sleeve has a peripheral wall defining an exterior surface and an interior passage sized to receive the tensile member;
crimping the crimp sleeve around the tensile member by an application of mechanical force so as to reduce its dimensions from a first size to a second, smaller size;
providing a cap having a bottom surface, a convex top surface, a central bore extending from the bottom surface to the top surface for receiving the crimp sleeve, wherein the cap includes a clamping structure disposed in the central bore; and
pressing the cap over the crimp sleeve such that the clamping structure deflects elastically and applies a clamping force to the crimp sleeve to secure the cap in place.

14. The method of claim 13 wherein the peripheral wall includes a circumferential array of lobes.

15. The method of claim 13 wherein the clamping structure comprises a split snap ring received in a groove formed in the central bore of the cap.

16. The method of claim 15 wherein the split snap ring includes a chamfered surface.

17. The method of claim 15 wherein the crimp sleeve includes at least one notch formed in the exterior surface thereof, sized to receive the split snap ring.

18. The method of claim 13 wherein the clamping structure comprises a circumferential array of resilient barbs which extend inward from the central bore.

19. The method of claim 13 wherein the clamping structure comprises the central bore having a shape complementary to the exterior surface of the crimp sleeve.

20. The method of claim 13 where the clamping structure comprises a snap ring received in a groove formed in the central bore, wherein the groove has a conical shape.
